(19) United States
(12) Patent Application Publication
(10) Pub. No.: US 2026/0283702 A1
(43) Pub. Date: Sep. 24, 2026

(54) FIDUCIAL AND FIDUCIAL RETENTION ON AN AIMER ARM, PRECISION AND LOADING FOR SURGICAL NAVIGATION SYSTEM

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Paul Alexander Torrie, Marblehead, MA (US); Geoffrey Ian Karasic, Raynham, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/475,979

(22) PCT Filed: Apr. 30, 2024
(86) PCT No.: PCT/US2024/027007

§ 371 Date: Oct. 16, 2025

Related U.S. Application Data
(60) Provisional application No. 63/468,159, filed on May 22, 2023.

(51) Int. Cl. A61B 34/20; A61B 90/00

(52) U.S. Cl. CPC A61B 34/20; A61B 90/39; A61B 2034/2055; A61B 2090/3991

(57) ABSTRACT

A fiducial for surgical device includes a body configured to be coupled to a distal end of an aimer arm and an optically detectable portion on an exterior of the body. The optically detectable portion is configured to be optically tracked during computer-assisted surgery. The body is configured to receive the distal end of the surgical device to directly attach to and directly detach from the surgical device.

This application claims priority to and the benefit of U.S. Prov. Pat. App. No. 63/468,159, filed May 22, 2023, which is incorporated herein by reference in its entirety.

### BACKGROUND

### Field of the Disclosure

This disclosure relates in general to surgical navigation and, in particular, a system, method and apparatus for a fiducial and fiducial retention on an aimer arm, precision and loading for a surgical navigation system.

### Description of the Prior Art

In computer-assisted arthroscopic surgery, fiducial blocks or fiducials with sensitive laser marks are used to provide a coordinate system to localize instruments and anatomy. The fiducials are attached to an aimer arm to allow the user to navigate their planned drill trajectories, which can be done in conjunction with software on the camera control unit.

Legacy aimer arms are reusable to reduce cost and waste. To avoid damage to the laser marks, and to avoid cleaning and sterilization issues associated with the laser marks, single use disposable fiducials also are used. Although these conventional solutions are workable, improvements in attaching fiducials to aimer arms continue to be of interest.

### SUMMARY

In one version, a fiducial for surgical navigation comprising a body has a length L, a width W, a height H and a hollow cavity with a cavity length, cavity width and cavity height, the body has an optically detectable portion configured to be optically tracked, and the body has a tab that is flexible relative to the body, the tab is configured to engage a recess in an aimer arm.

Another version includes a device for a navigation system for surgery with an aimer arm for external optical tracking. The device comprises a fiducial configured to be releasably coupled to the aimer arm adjacent to a distal end thereof in a sliding clearance fit, the fiducial comprises an optically detectable portion configured to be optically tracked, the fiducial has a flexure that is integral with the fiducial, the fiducial comprises an engaged position wherein the flexure is configured to engage and be retained on the aimer arm, a released position wherein the flexure is configured to disengage and be released from the aimer arm, and the engaged and released positions do not require a separate fastener to attach and detach, respectively.

In another version, a device for a navigation system for surgery can include an aimer arm for optical tracking for surgical navigation. The aimer arm can have a recess located adjacent a distal end. A fiducial can be releasably coupled to the aimer arm adjacent to the distal end in a sliding clearance fit. The fiducial can include an optically detectable portion configured to be optically tracked. The fiducial can have a flexure that is integral with the fiducial. The fiducial can have an engaged position where the flexure engages the recess such that the fiducial is retained on the aimer arm, and a released position where the flexure disengages the recess such that the fiducial is released from the aimer arm. The engaged and released positions may not require a separate fastener to attach and detach, respectively.

In still another version, a fiducial for surgical navigation has a body with a length L, a width W, a height H, and a hollow cavity with a cavity length, cavity width and cavity height. The body can have an optically detectable portion configured to be optically tracked. The body has an open front, open back and open bottom with a cross bar that spans side walls thereof. The cross bar and an inner surface of a top of the fiducial can define the cavity height.

A device for a navigation system for surgery with an aimer arm for external optical tracking, the device comprising a fiducial has an optically detectable portion configured to be optically tracked; and a compliance feature coupled to the fiducial, the fiducial comprises an engaged position wherein the compliance feature is configured to engage and be retained on the aimer arm, and a released position wherein the compliance feature is configured to disengage and be released from the aimer arm.

A device for a navigation system for surgery, the device comprising an aimer arm for external optical tracking and configured to be mounted to a base surgical component for surgical navigation, and the aimer arm has a position and orientation feature located adjacent a distal end thereof; a fiducial releasably coupled to the aimer arm adjacent to the distal end in a sliding clearance fit; and the fiducial comprises: an optically detectable portion configured to be optically tracked; and a compliance feature coupled to the fiducial, the fiducial comprises an engaged position wherein the compliance feature engages the position and orientation feature such that the fiducial is retained on the aimer arm, and a released position wherein the compliance feature disengages the position and orientation feature such that the fiducial is released from the aimer arm. A device for surgical navigation, the device comprising a fiducial comprising a body has a length L, a width W, a height H, a hollow cavity with a cavity length, cavity width and cavity height, the body has an optically detectable portion configured to be optically tracked, and the body has an open front, an open back and side walls; and an attachment mechanism configured to slidably and releasably couple the body to an aimer arm.

A device for a navigation system for robotic surgery with an aimer arm for external optical tracking, the device comprising a fiducial comprising an optically detectable portion configured to be optically tracked; and an attachment mechanism configured to slidably and releasably couple the fiducial to the aimer arm in a sliding clearance fit, the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

A device for a navigation system for surgery, the device comprising an aimer arm for external optical tracking and configured to be mounted to a base surgical component for surgical navigation, and the aimer arm has a proximal end and a distal end; a fiducial comprising an optically detectable portion configured to be optically tracked; and an attachment mechanism configured to slidably and releasably couple the fiducial to the aimer arm in a sliding clearance fit, the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

A device for a navigation system for robotic surgery, the device comprising a fiducial configured to be releasably coupled to an aimer arm with an attachment mechanism in a sliding clearance fit, the fiducial comprises an optically detectable portion configured to be optically tracked, and the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

A device for a navigation system for robotic surgery, the device comprising an aimer arm for external optical tracking and configured to be mounted to a base surgical component to for surgical navigation, and the aimer arm has a proximal end and a distal end; and a fiducial releasably coupled to the aimer arm with an attachment mechanism, the fiducial comprises an optically detectable portion configured to be optically tracked, and the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

A fiducial for surgical navigation, the fiducial comprising a body has a length L, a width W, a height H, a hollow cavity with a cavity length, cavity width and cavity height, the body has an optically detectable portion configured to be optically tracked, and the body has an open front, open back, an open bottom and sidewalls that can flexibly deform.

A fiducial for surgical navigation, the fiducial comprising a body has a length L, a width W, a height H and a hollow cavity with a cavity length, cavity width and cavity height, the body has an optically detectable portion configured to be optically tracked, and the body has a tab that is flexible relative to the body, the tab is configured to engage an aimer arm.

Still other embodiments are disclosed elsewhere herein and in the drawings.

### BRIEF DESCRIPTION OF THE DRAWINGS

Before one or more embodiments are described in detail, one skilled in the art will appreciate that they are not limited in its application to the details of construction, the arrangements of components, and the arrangement of steps set forth in the following detailed description or illustrated in the drawings and descriptions. Rather, they are capable of other embodiments and of being practiced or being carried out in various ways. Also, it is to be understood that the phraseology and terminology used herein is for the purpose of description and should not be regarded as limiting.

FIG. 1 is a perspective view of an embodiment of a fiducial mounted to an aimer arm.

FIG. 2 is a perspective view of an embodiment of the fiducial and aimer arm of FIG. 1 in operation.

FIG. 3 is an enlarged perspective view of an embodiment of a portion of the fiducial and aimer arm of FIG. 1, with the fiducial shown transparently for clarity.

FIG. 4 is a sectional view of the embodiment of FIG. 3.

FIG. 5 is a perspective view of an embodiment of a fiducial.

FIG. 6 is another perspective view of the embodiment of FIG. 5.

FIG. 7 is a perspective view of an embodiment of an aimer arm.

FIG. 8 is a perspective view of another embodiment of a fiducial and aimer arm prior to an engaged position.

FIG. 9 is a perspective view of the embodiment of FIG. 8 in the engaged position.

FIG. 10 is a perspective view of another embodiment of an aimer arm and a fiducial.

FIG. 11 is a perspective view of the embodiment of the fiducial and aimer arm of FIG. 10 in operation.

FIG. 12 is an enlarged perspective view of an embodiment of a portion of the fiducial and aimer arm.

FIG. 13 is a sectional perspective view of an embodiment of a portion of the fiducial and aimer arm.

FIG. 14 is a side view of a portion of an embodiment of the aimer arm and biasing force on the fiducial and resultant motion of the fiducial, with the fiducial not shown for clarity.

FIG. 15 is a bottom perspective view of another embodiment of the fiducial and aimer arm.

FIG. 16 is a side perspective view of still another embodiment of the fiducial and aimer arm.

FIG. 17 is a sectional side perspective view of the embodiment of FIG. 16.

FIG. 18 is a side perspective view of an alternate embodiment of the fiducial and aimer arm.

FIG. 19 is a bottom perspective view of another alternate embodiment of the fiducial and aimer arm.

FIG. 20 is a bottom sectional perspective view of the embodiment of FIG. 19.

FIG. 21 is another bottom sectional perspective view of the embodiment of FIG. 19, shown prior to deflection of a torsion latch.

FIG. 22 is another bottom sectional perspective view of the embodiment of FIG. 19, shown with the torsion latch deflected.

FIG. 23 is a side perspective view of an embodiment of a fiducial and aimer arm, prior to assembly.

FIG. 24 is a side perspective view of the embodiment of FIG. 23, shown partially assembled.

FIG. 25 is a side perspective view of the embodiment of FIG. 23, shown assembled.

FIG. 26 is a side perspective view of another embodiment of a fiducial and aimer arm.

FIG. 27 is a side perspective view of an embodiment of an attachment cord for a fiducial.

FIG. 28 is an end perspective view of another embodiment of a fiducial and attachment cord.

FIG. 29 is a side perspective view of another embodiment of an attachment cord for a fiducial.

FIG. 30 is a sectional top view of a portion of an embodiment of an attachment cord that demonstrates a mechanical deformation option.

FIG. 31 is a side perspective view of still another embodiment of an attachment cord and fiducial.

FIG. 32 is a side perspective view of yet another embodiment of an attachment cord and fiducial.

FIG. 33 is a perspective view of an alternate embodiment of a fiducial.

FIGS. 34A-34F are sectional side views of an embodiment of a fiducial and aimer arm during a process of assembly.

FIG. 35 is an enlarged view of FIG. 34D.

FIG. 36 is a sectional perspective view, similar to FIG. 34F.

FIG. 37 is a front perspective view of another embodiment of a fiducial and aimer arm, with the aimer arm shown transparent for clarity.

FIG. 38 is another front perspective view of the embodiment of FIG. 37.

FIG. 39 is a front perspective view of another embodiment of a fiducial.

FIG. 40 is a front view of the embodiment of FIG. 39.

FIG. 41 is a bottom perspective view of another embodiment of a fiducial.

FIG. 42 is an exploded, perspective view of another embodiment of a fiducial and aimer arm.

FIG. 43 is a sectional side view of the embodiment of FIG. 42 shown partially assembled.

FIG. 44 is a perspective view of the embodiment of FIG. 42 shown partially assembled.

FIG. 44 is a perspective view of the embodiment of FIG. 42 shown partially assembled.

FIG. 45 is a transparent, perspective view of the embodiment of FIG. 42 shown assembled.

FIG. 46 is a perspective view of yet another alternate embodiment of a fiducial and aimer arm.

FIG. 47 is a side view of the embodiment of FIG. 46.

FIG. 48 is a front view of the embodiment of FIG. 46.

### DETAILED DESCRIPTION

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings.

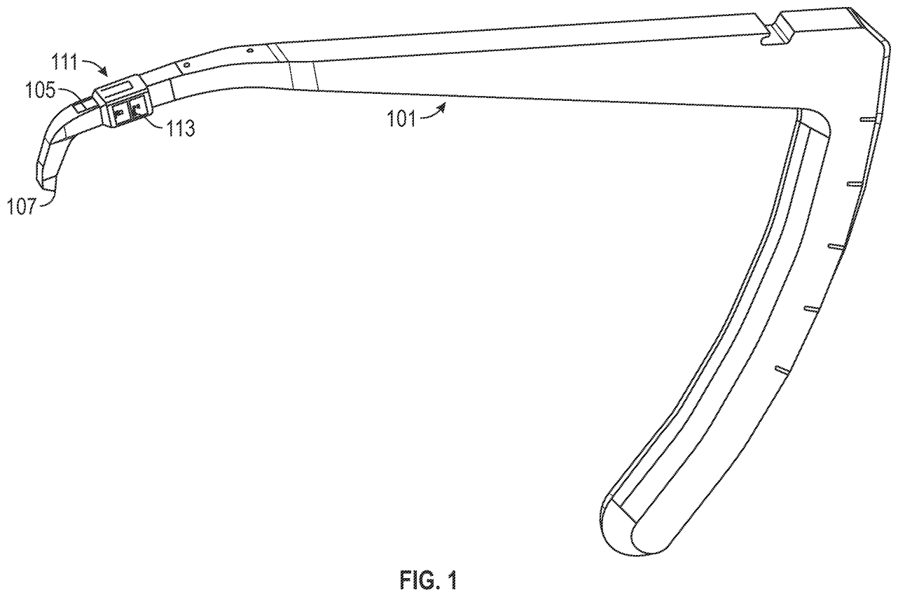

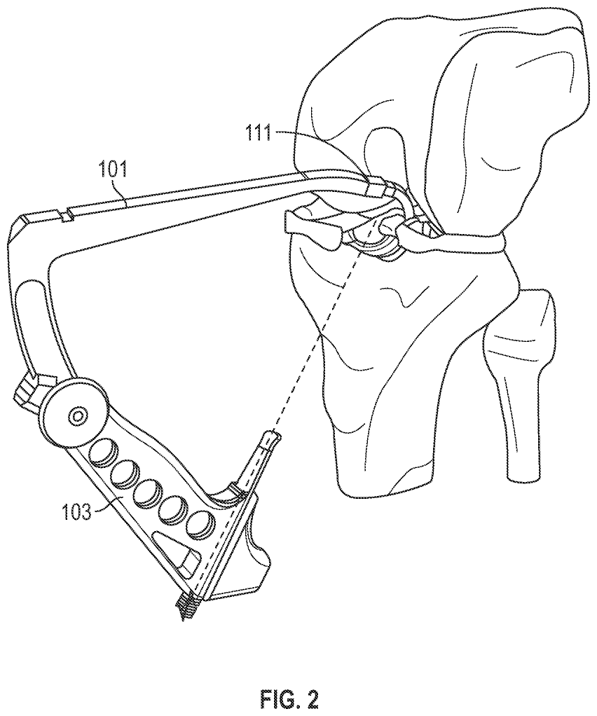

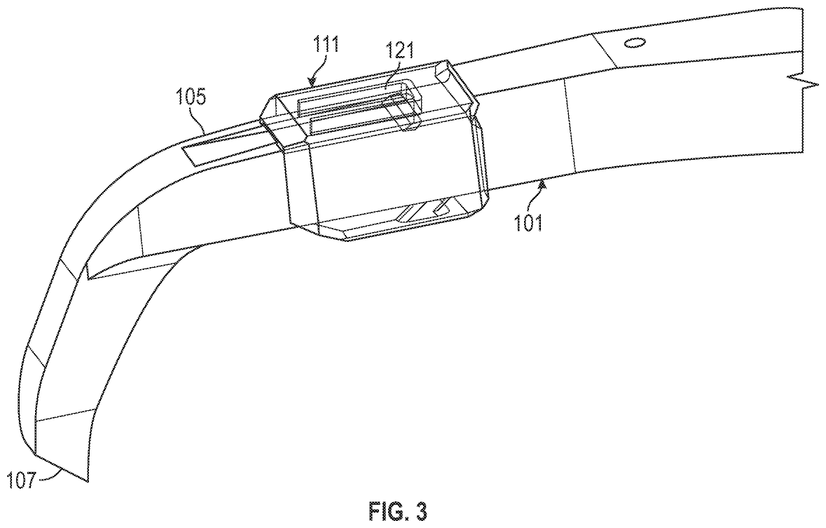

Referring to FIGS. 1-9, embodiments of a device for a navigation system for robotic and/or computer-assisted surgery are disclosed. For example, the device can be used in conjunction with an aimer arm 101 for external optical tracking. The aimer arm 101 can be configured to be mounted to a base surgical component 103 (FIG. 2) for surgical navigation. The aimer arm 101 can have a recess 105 located adjacent to a distal end 107 thereof.

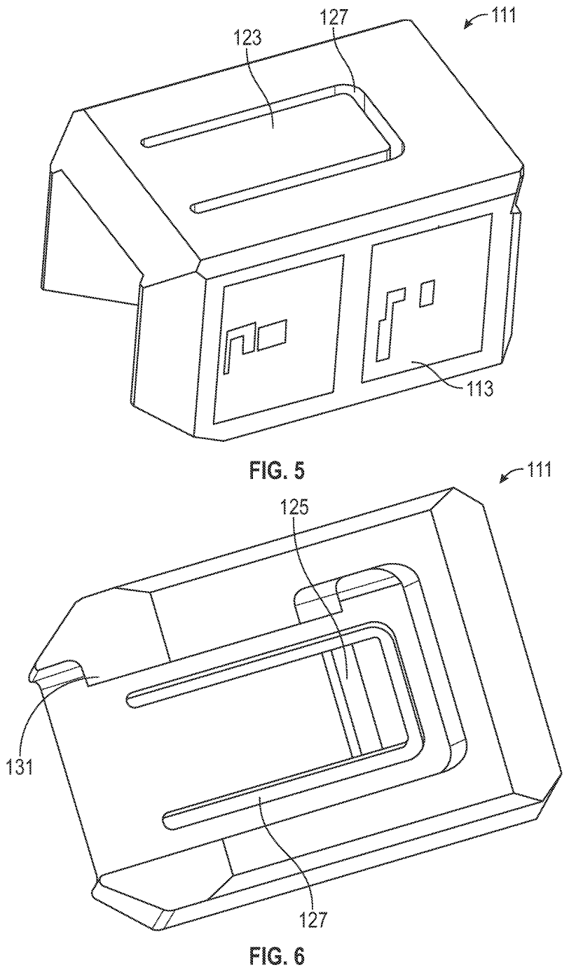

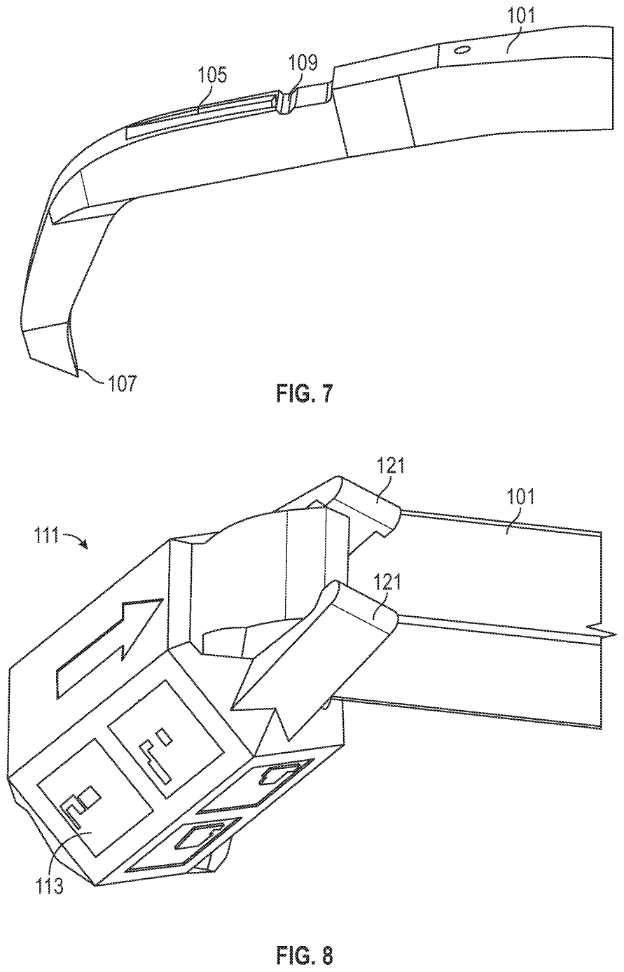

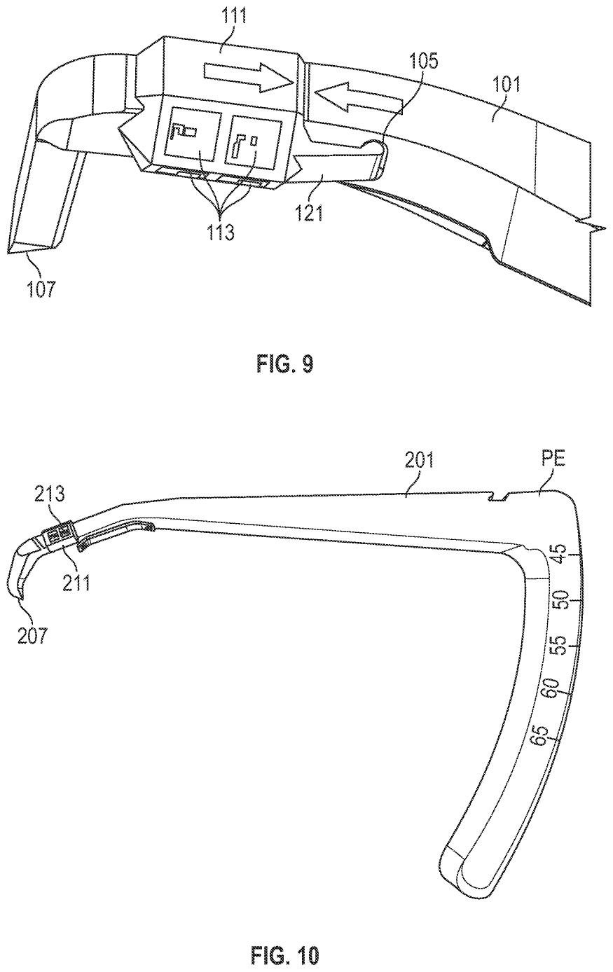

Embodiments of a fiducial 111 can be releasably coupled to the aimer arm 101 adjacent to the distal end 107 in a sliding clearance fit. The fiducial 111 can include an optically detectable portion 113 (FIGS. 8-9) that can be optically tracked. The fiducial 111 can have a flexure 121 that is integral with the fiducial 111. The fiducial 111 has an engaged position (FIGS. 1-4 and 9) wherein the flexure 121 engages the recess 105 such that the fiducial 111 is retained on the aimer arm 101. The fiducial also has a released position (FIGS. 5-8) wherein the flexure 121 disengages the recess 105 such that the fiducial 111 is released from the aimer arm 101. In some embodiments, the engaged and released positions do not require a separate fastener to attach and detach, respectively.

In one version, the aimer arm 101 does not include a separate latch arm for engaging the fiducial 111, such that the fiducial 111 directly attaches to and directly detaches from the aimer arm 101.

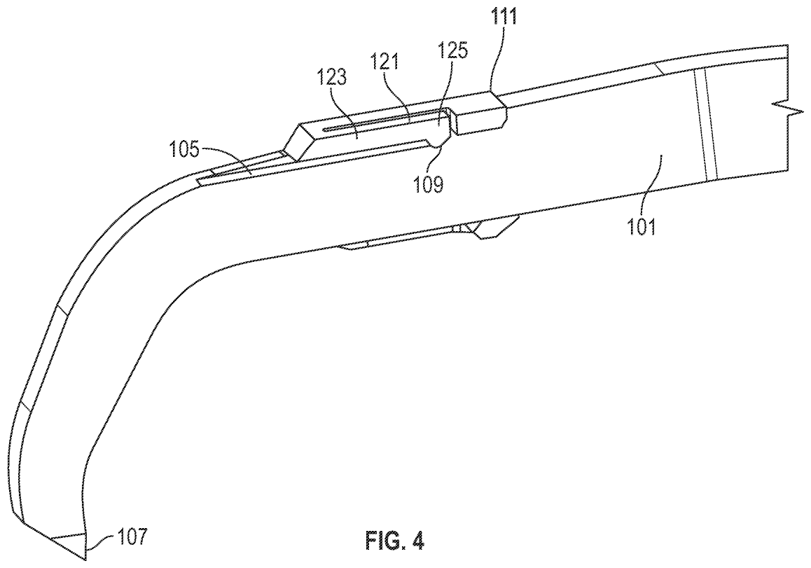

In some examples, the recess 105 in the aimer arm 101 comprises an elongated slot and a dimple 109 (FIGS. 4 and 7). As shown in FIGS. 4-6, the flexure 121 can comprise a tab or cantilevered tab 123 with a boss 125. The fiducial 111 can include a slot relief 127 around the cantilevered tab 123. The slot relief 127 can be on three sides of the cantilevered tab. The boss 125 can slidingly engage the elongated slot of the recess 105 before mating with the dimple 109 in the engaged position. Embodiments of the cantilevered tab 123 can be located within a central portion of the fiducial 111 and it can be flush with an exterior of the fiducial 111 such that an outer perimeter, form factor, outer envelope or outer shape of the fiducial 111 is unaltered by the cantilevered tab 123.

Other embodiments of the fiducial 111 can comprise a flange 131 opposite the flexure 121. The flange 131 can engage a surface of the aimer arm that is opposite the recess 105. The flange 131 can extend inward from a sidewall of the fiducial 111. In some versions, the flange 131 consists of a single flange such that the fiducial is asymmetrical.

In some examples (FIGS. 8 and 9), the recess 105 in the aimer arm 101 comprises at least two recesses 105, and the flexure 121 on the fiducial 111 comprises at least two flexures. The recesses 105 can be located on opposite sides of the aimer arm 101, and the flexures 121 can be located on opposite sides of the fiducial 111. The flexures 121 can extend away from the fiducial 111 and are configured to protrude away from the distal end 107 of the aimer arm 101.

Versions of the fiducial 111 can comprise an outer shape that is rectangular or hexagonal. The fiducial 111 has a length, width and height, such that a ratio of length to width is at least 2:1, and a ratio of length to height is at least 2:1, in some embodiments.

Referring now to FIGS. 10-22, other embodiments of a device for a navigation system for robotic and/or computer-assisted surgery are shown. For example, the device can include an aimer arm 201 for internal optical tracking and it is configured to be mounted to a base surgical component 203 for surgical navigation. The aimer arm 201 has a position and orientation feature 206 located adjacent a distal end 207.

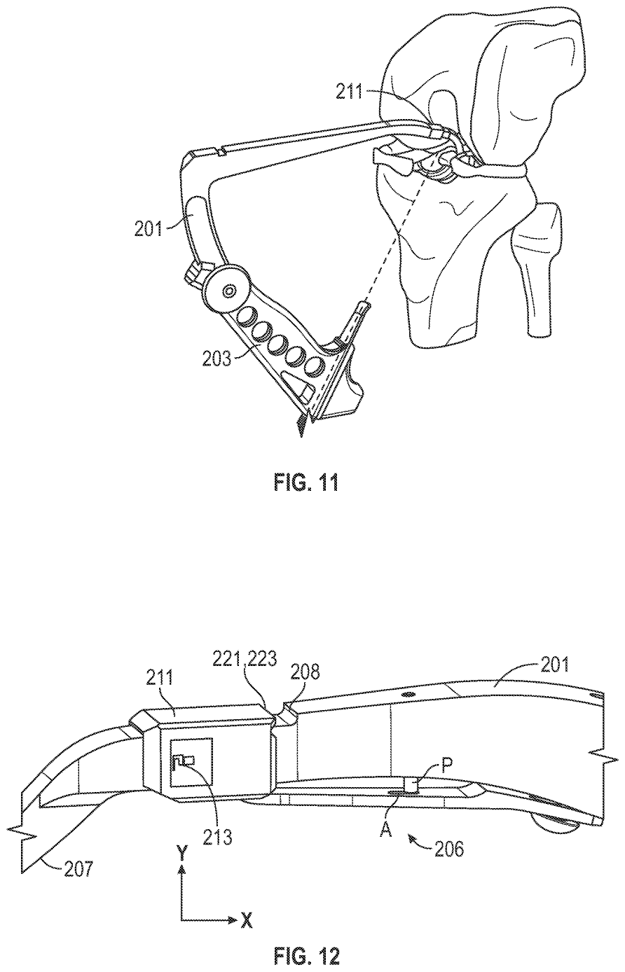

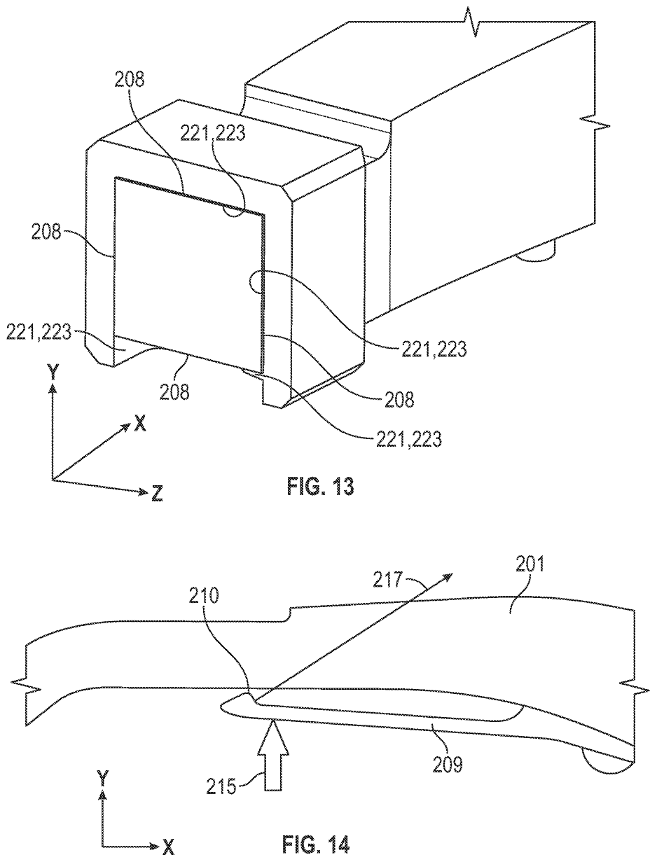

A fiducial 211 can be releasably coupled to the aimer arm 201 adjacent to the distal end 207, for example, in a sliding clearance fit. The fiducial 211 comprises an optically detectable portion 213 configured to be optically tracked. The fiducial 211 also has a compliance feature 221 (FIGS. 12-13) that can be integrally formed with the fiducial 211. The fiducial 211 has an engaged position (FIGS. 10-11) where the compliance feature 221 engages the position and orientation feature 206 such that the fiducial 211 is retained on the aimer arm 201. The fiducial 211 also has a released position (FIGS. 12-14) where the compliance feature 221 disengages the position and orientation feature 206 such that the fiducial 211 is released from the aimer arm 201.

Embodiments of the compliance feature 221 and the position and orientation feature 206 can comprise components that are complimentary in shape. As shown in FIGS. 12-13, the position and orientation feature 206 can comprise a hard stop and surfaces 208, and the compliance feature 221 comprises fiducial block surfaces 223. In one example, at least two of the fiducial block surfaces 223 are perpendicular to each other. In some versions, the compliance feature 221 and the position and orientation feature 206 are configured to restrain the fiducial 211 in six degrees of freedom, including both +and −x-directions, both +and −y-directions and both +and −z-directions.

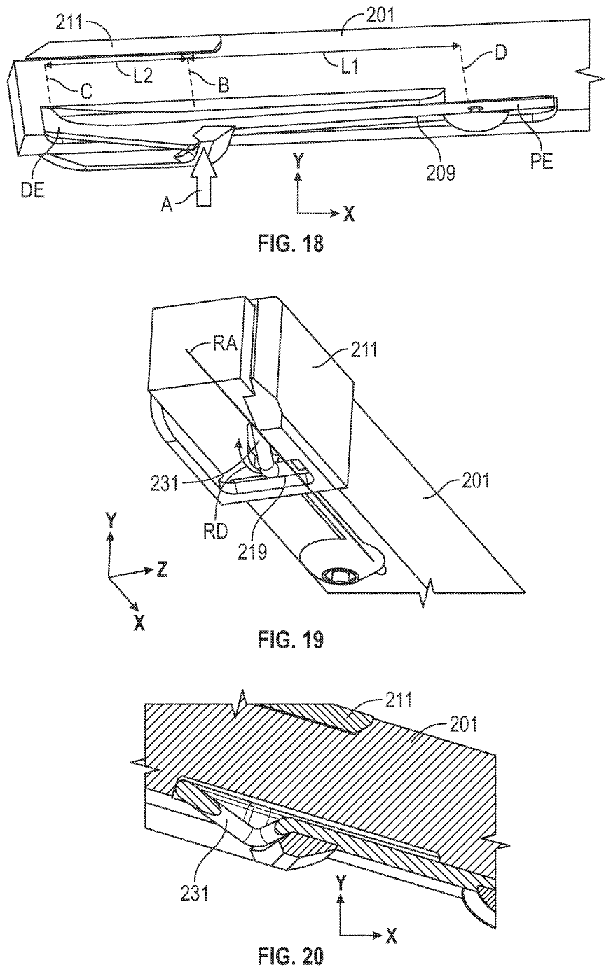

At least one of the compliance feature 221 and the position and orientation feature 206 can comprise a latch. For example, the latch 209 (FIG. 14) can be located on the aimer arm 201. The latch 209 can be mounted to and extend from the aimer arm 201, such as from an exterior of the aimer arm 201. The latch 209 can provide a biasing force 215 with a resultant motion 217 of the fiducial 211 being toward at least one surface of the aimer arm 201 and away from the distal end 207 of the aimer arm 201. The latch 209 may include a latch ramp 210 that engages a fiducial ramp 219 (see FIGS. 17 and 19) of the fiducial 211 that is complementary in shape to the latch ramp 210. The latch ramp 210 and the fiducial ramp 219 may comprise edges or non-planar surfaces (see, e.g., FIG. 35). The latch 209 can extend from the aimer arm 201 onto an exterior portion of the fiducial 211 and contact an outer surface (e.g., the latch ramp 219) of the fiducial 211.

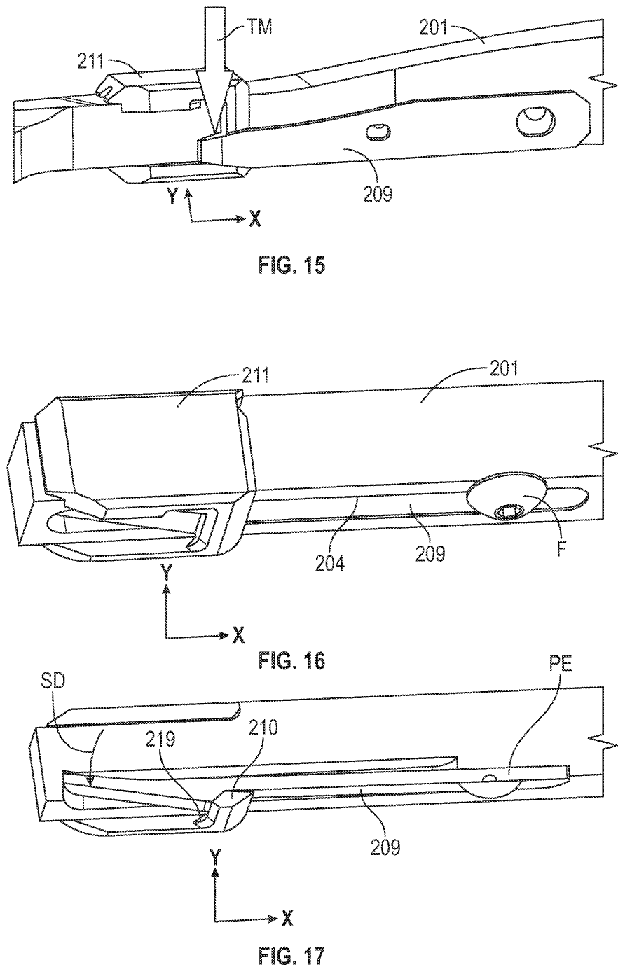

As shown in FIG. 15, the latch 209 can be offset from a plane of symmetry for at least one of the fiducial 211 and aimer arm 201 to provide asymmetry and a twisting moment TM on the object it contacts. For example, the latch 209 can extend from the aimer arm 201 and provide the twisting moment TM on the fiducial 211. Alternatively, the latch 209 can be cantilevered on at least one of the fiducial 211 (e.g., FIGS. 8-9, like flexures 121) and the aimer arm 201. For example, the latch 209 can be located in a slot 204 (FIG. 16) in the aimer arm 201 and is mounted thereto with a fastener F. In one version, the engaged and released positions do not require a separate fastener to attach and detach, respectively, the fiducial 211 with respect to the aimer arm 201. In some versions, the latch 209 can extend from the aimer arm 201 into an interior of the fiducial 211 and contact an inner portion of the fiducial 211. A recessed latch 209, such as this, is less like to catch soft tissue or bend during cleaning. Alternatively, the latch can be integrally formed with the fiducial 211 (e.g., FIGS. 8-9, again like flexures 121).

As shown in FIG. 17, the latch 209 can consist a single cantilever at a proximal end PE thereof, such that the distal end DE of the latch 209 is sprung down, as shown by arrow SD. Alternatively, the latch 209 can comprise a first cantilever D (FIG. 18) at the proximal end PE and a second cantilever C at the distal end DE, such that the latch 209 is deflected as shown by arrow A toward B. In one version, a first cantilever length L1 differs from a second cantilever length L2 such that the first and second cantilever lengths L1, L2 are asymmetrical. In still another alternative, the latch 209 can comprise a pair of cantilevered latches (like FIGS. 8-9). The pair of cantilevered latches can be located side by side, in one version.

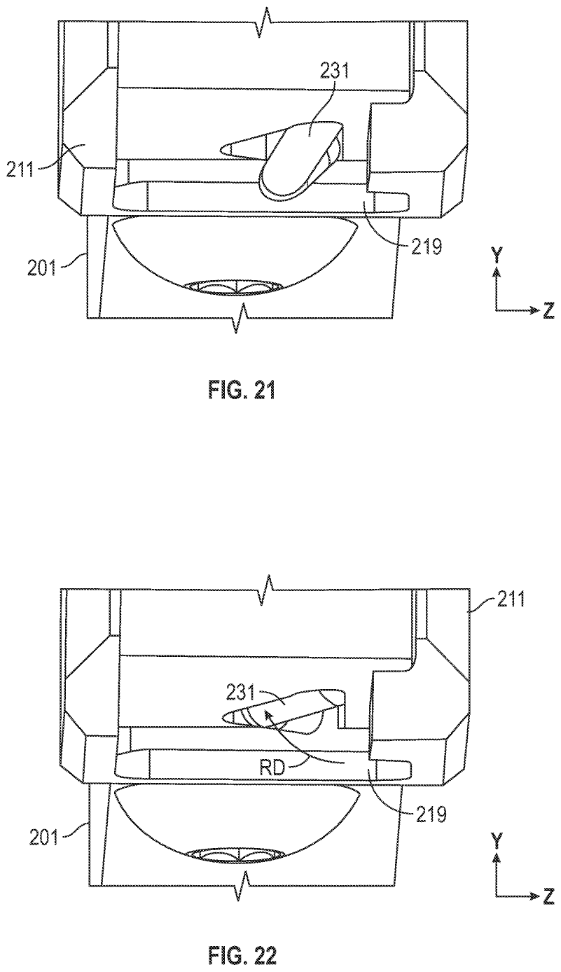

FIGS. 19-22 depict another alternative for the latch as at least one torsional spring 231. The torsional spring 231 comprises a rotational axis RA, a recessed position (FIGS. 19 and 22) within the aimer arm 201, and extended position (FIGS. 20 and 21) extending away from the aimer arm 201. The torsional spring 231 is configured to rotationally deflect the fiducial 211 toward the aimer arm 201. As shown in FIG. 20, the torsional spring 231 can engage an internal fiducial ramp 235 on an interior of the fiducial 211.

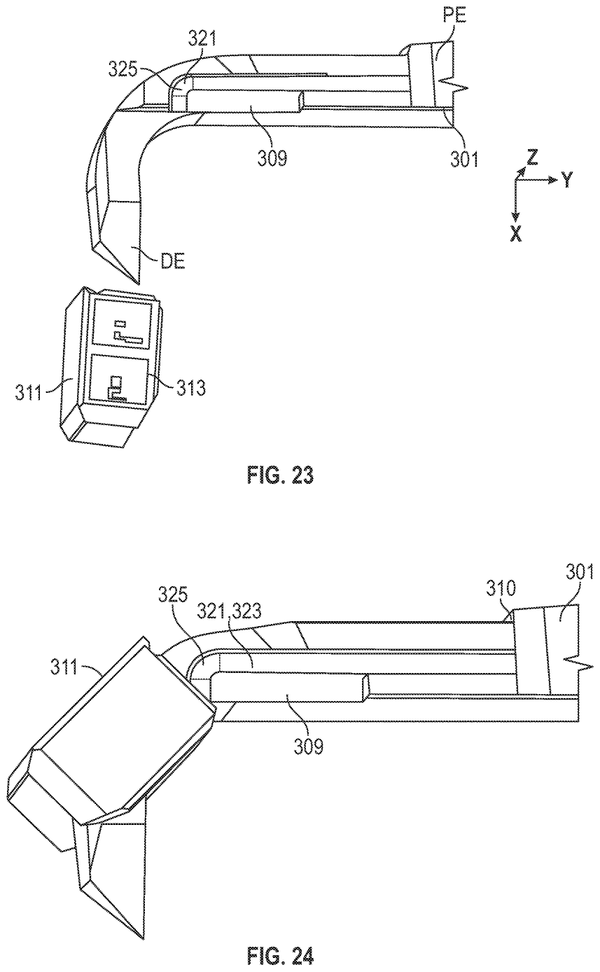

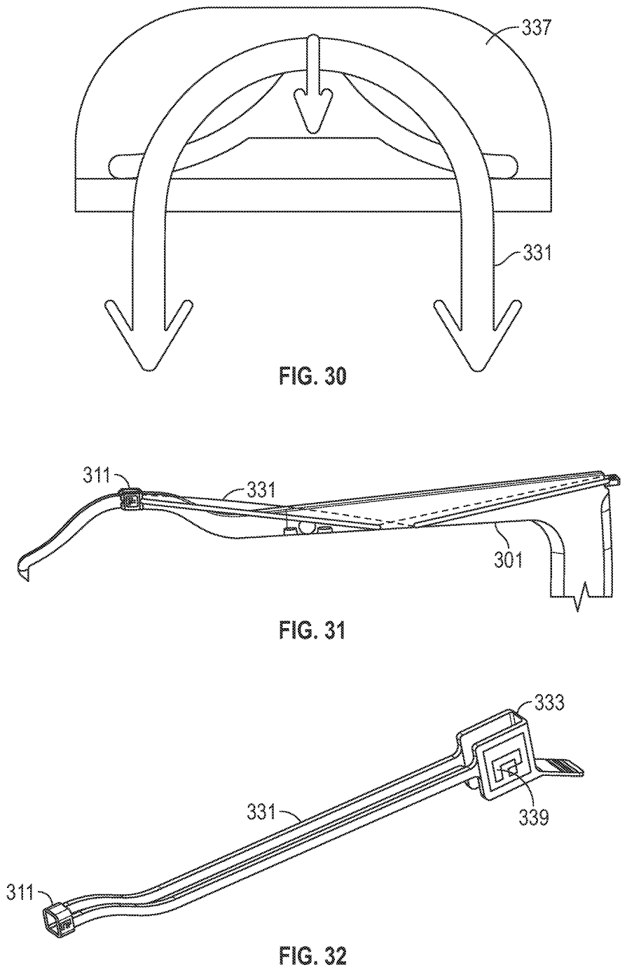

Other embodiments of a device for a navigation system for robotic and/or computer-navigated surgery are disclosed. For example, the device can include many, if not all, of the features disclosed in FIGS. 10-13 and their descriptive paragraphs. In addition, these embodiments can include other features and components, such as those shown in FIGS. 23-32. As shown in FIG. 23, an aimer arm 301 can have a proximal end PE and a distal end DE. A fiducial 311 can comprise an optically detectable portion 313 configured to be optically tracked.

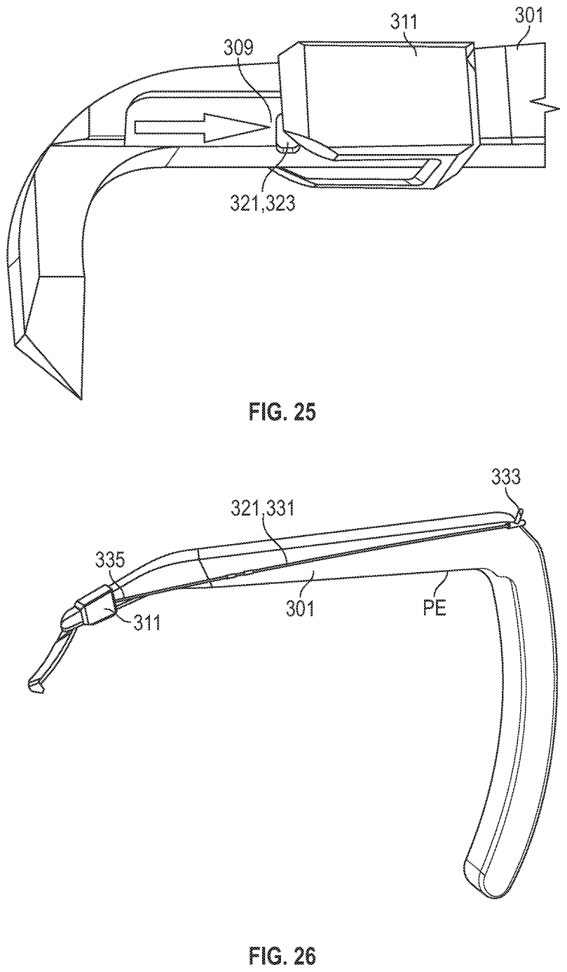

In addition, an attachment mechanism 321 can be configured to slidably and releasably couple the fiducial 311 to the aimer arm 301 in a sliding clearance fit. The fiducial 311 comprises an engaged position (FIG. 25) wherein the attachment mechanism 321 engages both the aimer arm 301 and the fiducial 311 such that the fiducial 311 is retained on the aimer arm 301, and a released position (FIGS. 23 and 24) wherein the attachment mechanism 321 disengages such that the fiducial 311 is released from the aimer arm 301.

In some embodiments, the attachment mechanism 321 comprises a retaining wire 323. The retaining wire 323 can be in a recess 309 of the aimer arm 301. The retaining wire 323 can extend for a limited length of the aimer arm 301 and can have a bend 325 at a distal end thereof. The retaining wire 323 can have an unlocked position (FIG. 23) that corresponds to the released position of the fiducial 311, and a locked position (FIG. 25) that corresponds to the engaged position of the fiducial 311. The retaining wire 323 can move with the fiducial 311 when the fiducial 311 moves between the engaged and released positions. In the engaged position, the retaining wire 323 can mechanically interfere with the fiducial 311 to secure it against a hard stop 310 (FIG. 24) on the aimer arm 301. The retaining wire 323 can impart a biasing force against the fiducial 311 to ensure contact with the hard stop 310. The retaining wire 323 can act as a wedge between the fiducial 311 and the aimer arm 301.

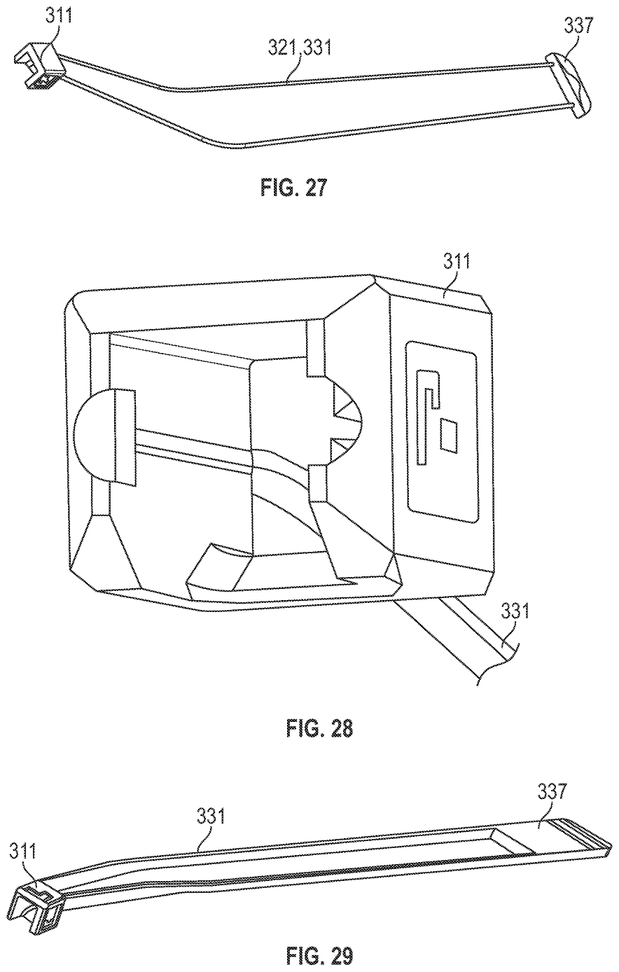

As shown in FIGS. 26-27, the attachment mechanism 321 may comprise a strap 331 that couples the fiducial 311 to the aimer arm 301 in the engaged position. The strap 331 can be removable from the aimer arm 301. The strap 331 can be elastic or inelastic. The strap 331 can have one or two strap sides, in some versions. The strap 331 can have a strap proximal end 333 that is configured to be coupled to or adjacent to the proximal end PE of the aimer arm 301, and a strap distal end 335 that is configured to be coupled to the fiducial 311. The strap 331 can be coupled to a finger tab 337 (FIGS. 27, 29 and 30) that can be secured to or adjacent to the proximal end PE of the aimer arm 301. In one example, the strap 331 is molded and integrally formed with the fiducial 311 from a same material. As shown in FIG. 28, the strap 331 and the fiducial 311 can be molded together from different materials.

In the engaged position, each of the two strap sides of strap 331 can extend along only one side of the aimer arm 301 (FIG. 26). Alternatively, in the engaged position, each of the two strap sides of strap 331 can cross (FIG. 31) and extend along two or more sides of the aimer arm 301. As shown in FIG. 32, the strap 331 can comprise an additional fiducial 339 (FIG. 32) at or adjacent to the strap proximal end 333 such that, when the fiducial 311 is inside a body of a surgical patient, the additional fiducial 339 is visible from outside the body of the surgical patient.

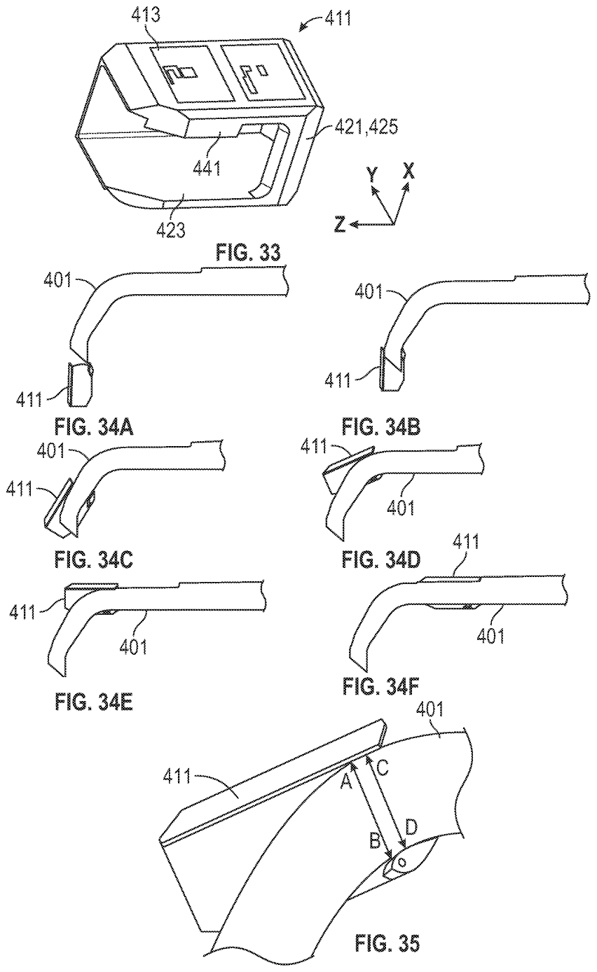

Still other embodiments of a device for a navigation system for robotic and/or computer-navigated surgery are disclosed. For example, these devices can include many, if not all, of the features disclosed in FIGS. 10-13 and their descriptive paragraphs. In addition, these embodiments can include other features and components, such as those shown in FIGS. 33-40.

As shown in FIGS. 33-34F, a fiducial 411 can be releasably coupled to an aimer arm 401 with an attachment mechanism 421 in a sliding clearance fit. The fiducial 411 has an optically detectable portion 413 configured to be optically tracked. The fiducial 411 has an engaged position (FIG. 34F) wherein the attachment mechanism 421 engages both the aimer arm 401 and the fiducial 411 such that the fiducial 411 is retained on the aimer arm 401. The fiducial also has a released position (FIGS. 33 and 34A-34E) wherein the attachment mechanism 421 disengages such that the fiducial 411 is released from the aimer arm 401.

Versions of the attachment mechanism 421 can comprise an open side 423 of the fiducial 411 and a cross bar 425 that spans the open side 423. The fiducial 411 can be open on its front and rear and, other than the open side 423 with the cross bar 425, is closed on every other side. As shown in FIG. 35, the fiducial 411 comprises an internal bore height A-B at the cross bar 425 that is greater than a height C-D of the aimer arm 401 at a bend in the aimer arm 401, such that edges E-F of the cross bar 425 are spaced apart from each other.

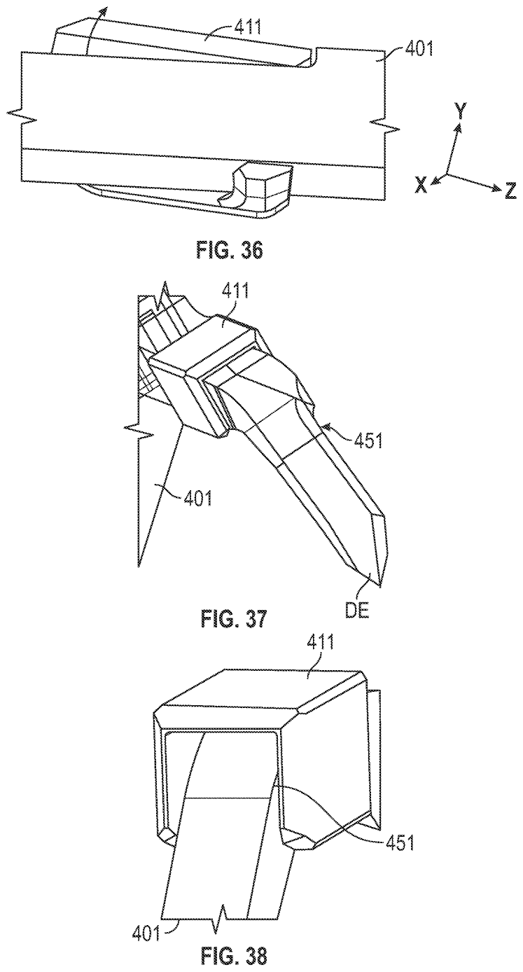

Referring again to FIG. 33, the fiducial 411 can comprise a partial shelf 441 for engaging a surface of the aimer arm 401. The partial shelf 441 can prevent rotational motion of the fiducial 411, as shown by arrow 443 in FIG. 36. The partial shelf 441 can extend inward from a sidewall of the fiducial 411. The partial shelf 441 can consist of a single partial shelf such that the fiducial 411 is asymmetrical, as shown. In one example, the partial shelf 441 is not in contact with or immediately adjacent to the cross bar 425.

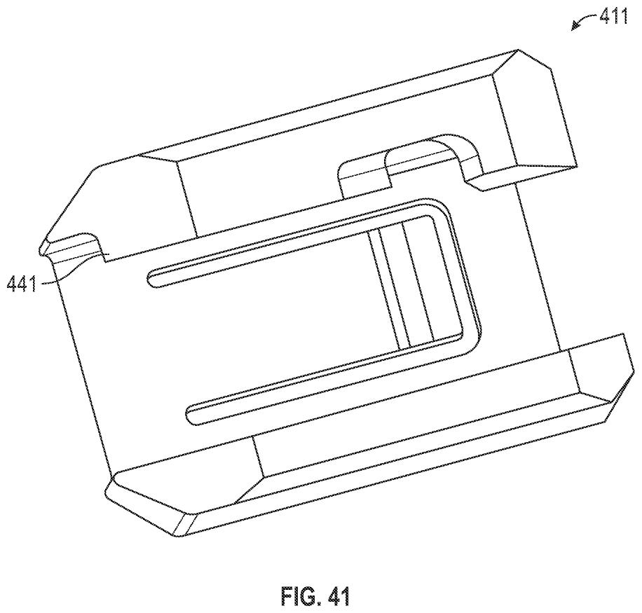

In some embodiments, the attachment mechanism 421 can comprise the combination of the open side 423 of the fiducial 411 and the partial shelf 441 that extends inward from the sidewall of the fiducial into the open side 423, such that the fiducial 411 does not comprise a cross bar 425 (FIG. 41). A length of the partial shelf 441 can be less than an overall length of the fiducial 411. In one version, the fiducial 411 is open on its front and rear and, other than the open side 423 with the partial shelf 441, is closed on every other side. In another version, the partial shelf 441 can comprise two partial shelves located opposite each other and extending toward each other.

As shown in FIGS. 37-38, the aimer arm 401 can have a shelf clearance 451 adjacent to the distal end DE for accommodating the partial shelf 441 of the fiducial 411 during installation. The shelf clearance 451 can be on only one side of the aimer arm 401 such that the aimer arm 401 is asymmetric. The fiducial 411 can be restrained in five degrees of freedom, including both +and −x-directions, both +and −y-directions and one z-direction.

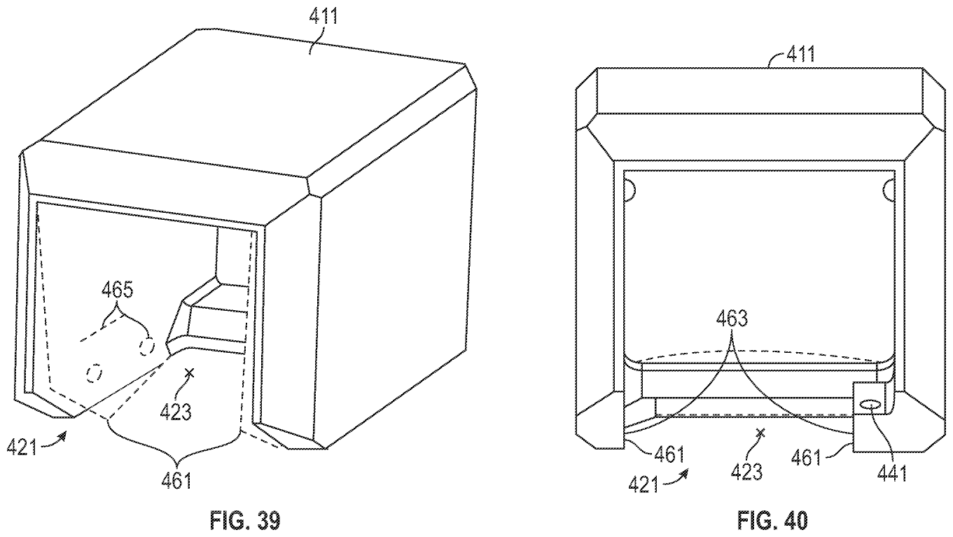

In FIGS. 39-40, an embodiment of the attachment mechanism 421 is depicted with inwardly-biased walls 461 to secure the fiducial 411 to the aimer arm 401 in the engaged position. In some examples, exposed edges 463 of the inwardly-biased walls 461 are spaced apart from each other by the open side 423 in the fiducial 411.

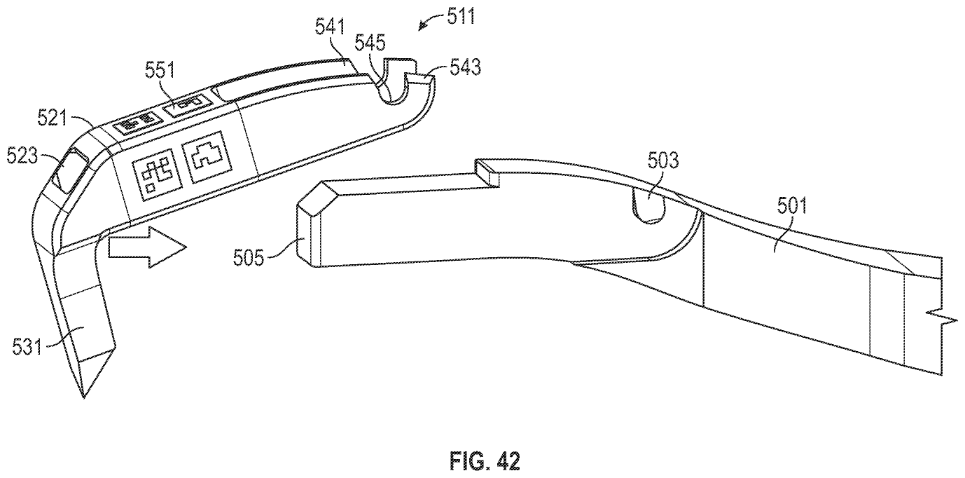

FIGS. 42-45 depict another embodiment of “whole tip” fiducial 511 and a complementary aimer arm 501. The fiducial 511 can be for a navigation system for robotic surgery, as described herein. The fiducial 511 can include a hollow body 521 configured to be coupled to an end 505 (e.g., distal end) of the aimer arm 501. The aimer arm 501 can insert into the hollow body 521. The end 505 can be blunt without an aimer tip or other aimable feature for computer-assisted surgery.

The hollow body 521 can include a pointer 531 that is integral with and extends from a distal end of the hollow body 521. Note that the pointer 531 is an aimable feature during computer-assisted surgery, rather than having the aimable feature as part of the aimer arm 501.

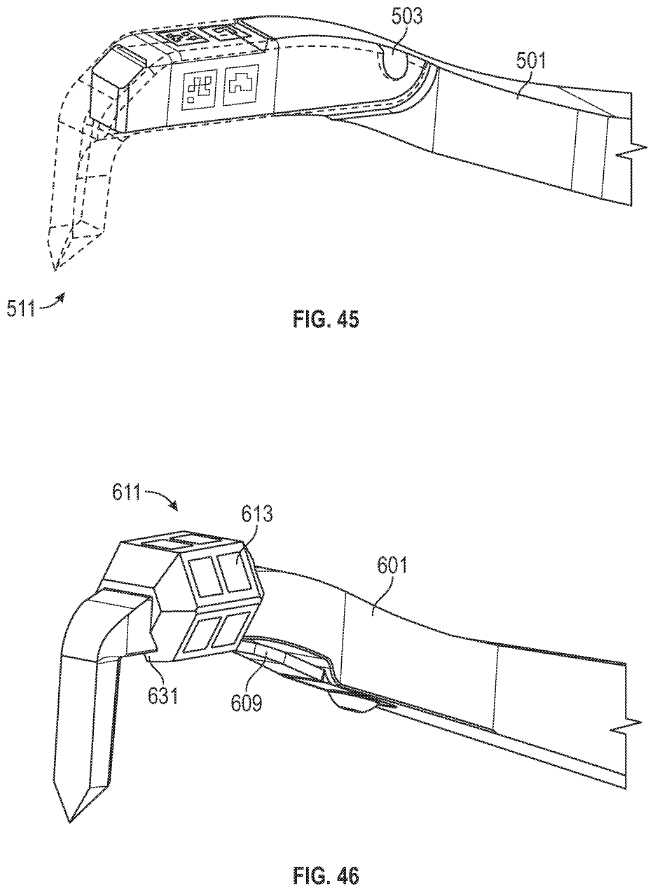

An engagement feature 541 is integral with a proximal end of the hollow body 521. The engagement feature 541 comprises an engaged position (FIG. 45-46) configured to engage the aimer arm 501 such that the fiducial 511 is retained on the aimer arm, and a released position (FIGS. 42-44) wherein the engagement feature 541 disengages the aimer arm 501 such that the hollow body 521 is released from the aimer arm 501. The hollow body 521 can slidingly snap into place in the engaged position.

An optically detectable portion 551 also is on an exterior of the hollow body 521. The optically detectable portion 551 is configured to be optically tracked during computer-assisted surgery, as described herein. A top of the hollow body 521 also can include the optically detectable portion 551.

The hollow body 521 and the engagement feature 541 are configured to directly attach to and directly detach from the aimer arm 501 without a separate retention component. The engagement feature comprises a cantilevered, elastic tab 543 with a recess 545 that is configured to engage, in the engaged position, a protrusion 503 on the aimer arm 501. In the engaged position, the fiducial 511 is substantially linearly and rotationally immobilized relative to the aimer arm 501 until the cantilevered, elastic tab 543 is released from the protrusion 503 to allow the released position. The engagement feature 541 can include a pair of the tabs 543 to engage opposite sides of the aimer arm 501.

In addition, the hollow body 521 can include a hole or aperture 523 (e.g., a through hole, blind hole, etc.) configured to receive the end of the aimer arm 501. The aperture 523 can be located between the pointer 531 and the optically detectable portion 551.

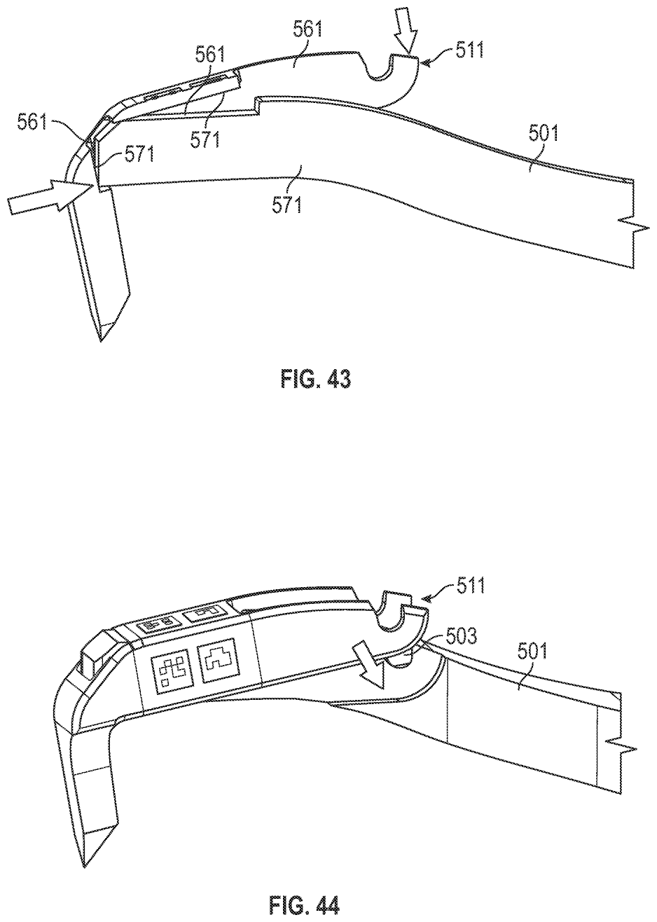

In addition, the hollow body 521 can include shoulders 561 (FIG. 43), such as surfaces, that can engage respective features 571 (e.g., surfaces) on the aimer arm 501. The fiducial 511 can rotate in a single plane to move from the released position to the engaged position.

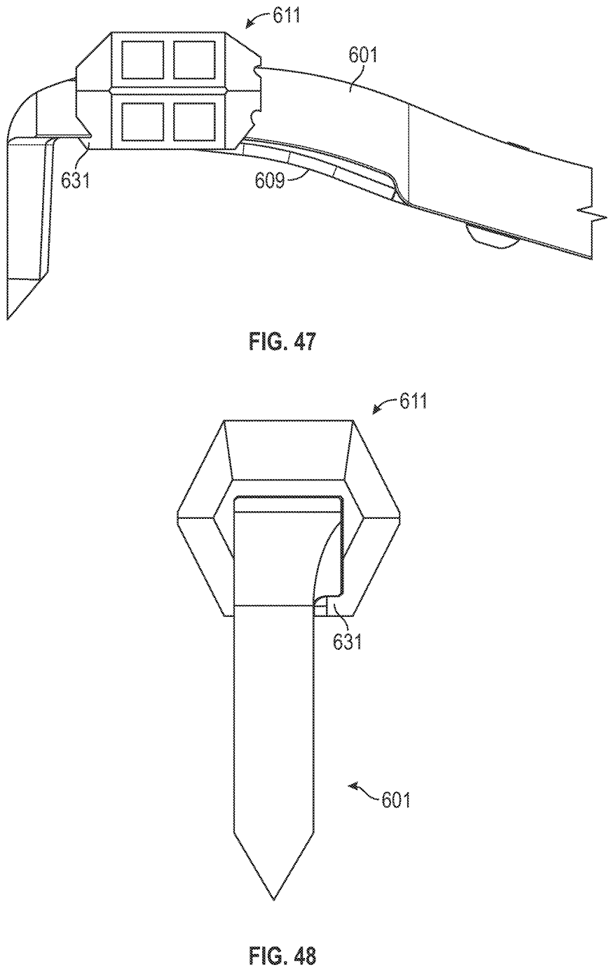

FIGS. 46-48 yet another alternative embodiment of an aimer arm 601 and fiducial 611. This embodiment incorporates features and elements described above for the other embodiments. For example, the fiducial 611 has some of the features described and shown in FIGS. 8-9. In addition, fiducial 611 can have a flange 631 for better retention, as described and shown in FIGS. 6 and 40.

Note that these fiducials can have almost any external shape. For example, although fiducial 611 is shown with a hexagonal appearance, it could be rectangular, pentagonal, etc., and even may be asymmetrical. It can be useful to have the optically detectable portions 613 on flat, planar surfaces of the fiducial 611.

Embodiments of the aimer arm 601 can include a spring, wire or latch 609 (or other engagement devices), such as those described and shown in FIGS. 14-24 Various embodiments are directed to an improved way of attaching an aimer tip fiducial to an aimer arm and, more particularly, to engagement means for securing an aimer tip fiducial to an aimer arm.

The engagement means can be on an end of the aimer arm. The engagement means can be one or more tangs, such as cantilevered tabs with a recess on each end of each tab. The tabs are elastically forced out when the aimer tip fiducial is attached (in one plane) to the aimer arm. The tabs then snap onto and are retained by protrusions in slots on the aimer arm. The slots can be complementary in shape to the shapes of the tabs to confirm correct installation and for positive retention. The drawings illustrate how the fiducial is installed and retained.

Any of the embodiments disclosed herein may use features or components from any other embodiment, with or without any limitations. Still other embodiments can include one or more of the following items.

1. A fiducial for surgical navigation, the fiducial comprising:
a body has a length L, a width W, a height H and a hollow cavity with a cavity length, cavity width and cavity height, the body has an optically detectable portion configured to be optically tracked, and the body has a tab that is flexible relative to the body, the tab is configured to engage a recess in an aimer arm.

2. The fiducial wherein the tab extends into the hollow cavity to reduce the cavity height.

3. The fiducial wherein the tab protrudes away from the body.

4. The fiducial wherein the tab comprises a plurality of tabs, each with a boss.

5. The fiducial wherein the tab is cantilevered relative to the body.

6. The fiducial wherein the cantilevered tab is located within a central portion of the fiducial and is flush with an exterior of the fiducial such that an outer perimeter of the fiducial is unaltered by the cantilevered tab.

7. The fiducial wherein the fiducial comprises a slot relief around the tab.

8. The fiducial wherein the slot relief is on three sides of the tab.

9. The fiducial wherein the fiducial comprises a flange opposite the tab for engaging a surface of the aimer arm that is opposite the recess.

10. The fiducial wherein the flange extends inward from a sidewall of the fiducial.

11. The fiducial wherein the flange consists of a single flange such that the fiducial is asymmetrical.

12. A device for a navigation system for surgery with an aimer arm for external optical tracking, the device comprising:
a fiducial configured to be releasably coupled to the aimer arm adjacent to a distal end thereof in a sliding clearance fit, the fiducial comprises an optically detectable portion configured to be optically tracked, the fiducial has a flexure that is integral with the fiducial, the fiducial comprises an engaged position wherein the flexure is configured to engage and be retained on the aimer arm, a released position wherein the flexure is configured to disengage and be released from the aimer arm, and the engaged and released positions do not require a separate fastener to attach and detach, respectively.

13. A device for a navigation system for surgery, the device comprising:
an aimer arm for optical tracking for surgical navigation, and the aimer arm has a recess located adjacent a distal end; and
a fiducial releasably coupled to the aimer arm adjacent to the distal end in a sliding clearance fit, the fiducial comprises an optically detectable portion configured to be optically tracked, the fiducial has a flexure that is integral with the fiducial, the fiducial comprises an engaged position wherein the flexure engages the recess such that the fiducial is retained on the aimer arm, a released position wherein the flexure disengages the recess such that the fiducial is released from the aimer arm, and the engaged and released positions do not require a separate fastener to attach and detach, respectively.

14. The device wherein the aimer arm does not include a separate latch arm for engaging the fiducial, such that the fiducial directly attaches to and directly detaches from the aimer arm.

15. The device wherein the recess comprises an elongated slot and a dimple, and the flexure comprises a cantilevered tab with a boss that engages the dimple in the engaged position.

16. The device wherein the cantilevered tab is located within a central portion of the fiducial and is flush with an exterior of the fiducial such that an outer perimeter of the fiducial is unaltered by the cantilevered tab.

17. The device wherein the dimple is in the elongated slot and the boss is configured to slidingly engage the elongated slot before mating with the dimple in the engaged position.

18. The device wherein the fiducial comprises a slot relief around the cantilevered tab.

19. The device wherein the slot relief is on three sides of the cantilevered tab.

20. The device wherein the fiducial comprises a flange opposite the flexure for engaging a surface of the aimer arm that is opposite the recess.

21. The device wherein the flange extends inward from a sidewall of the fiducial.

22. The device wherein the flange consists of a single flange such that the fiducial is asymmetrical.

23. The device wherein the recess in the aimer arm comprises at least two recesses, and the flexure on the fiducial comprises at least two flexures.

24. The device wherein the recesses are located on opposite sides of the aimer arm, and the flexures are located on opposite sides of the fiducial.

25. The device wherein the flexures extend away from the fiducial and are configured to protrude away from the distal end of the aimer arm.

26. The device wherein the fiducial comprises an outer shape that is rectangular or hexagonal.

27. The device wherein the fiducial has a length, width and height, a ratio of length to width is at least 2:1, and a ratio of length to height is at least 2:1.

28. A fiducial for surgical navigation, the fiducial comprising:
a body has a length L, a width W, a height H, a hollow cavity with a cavity length, cavity width and cavity height, the body has an optically detectable portion configured to be optically tracked, and the body has an open front, open back and open bottom with a cross bar that spans side walls thereof, wherein the cross bar and an inner surface of a top of the fiducial define the cavity height.

29. The fiducial wherein the cross bar comprises an internal ramp.

30. The fiducial wherein the cross bar comprises an external ramp.

31. The fiducial wherein the fiducial comprises an external convex feature comprising a hard stop that is configured to abut an aimer arm.

32. A device for a navigation system for surgery with an aimer arm for external optical tracking, the device comprising:
a fiducial has an optically detectable portion configured to be optically tracked; and
a compliance feature coupled to the fiducial, the fiducial comprises an engaged position wherein the compliance feature is configured to engage and be retained on the aimer arm, and a released position wherein the compliance feature is configured to disengage and be released from the aimer arm.

33. The device wherein the compliance feature is integrally formed with the fiducial.

34. The device wherein the compliance feature is not integrally formed with the fiducial and is mounted to the fiducial.

35. A device for a navigation system for surgery, the device comprising:
an aimer arm for external optical tracking and configured to be mounted to a base surgical component for surgical navigation, and the aimer arm has a position and orientation feature located adjacent a distal end thereof;
a fiducial releasably coupled to the aimer arm adjacent to the distal end in a sliding clearance fit; and the fiducial comprises:
an optically detectable portion configured to be optically tracked; and
a compliance feature coupled to the fiducial, the fiducial comprises an engaged position wherein the compliance feature engages the position and orientation feature such that the fiducial is retained on the aimer arm, and a released position wherein the compliance feature disengages the position and orientation feature such that the fiducial is released from the aimer arm.

36. The device wherein the compliance feature is integrally formed with the fiducial.

37. The device wherein the compliance feature is not integrally formed with the fiducial and is attached to the fiducial.

38. The device wherein the aimer arm comprises a pin that extends therefrom into an aperture of the position and orientation feature to limit twist of the position and orientation feature.

39. The device wherein the compliance feature and the position and orientation feature comprise components that are complimentary in shape.

40. The device wherein position and orientation feature comprises a hard stop and surfaces, and the compliance feature comprises fiducial block surfaces.

41. The device wherein at least two of the fiducial block surfaces are perpendicular to each other.

42. The device wherein the compliance feature and the position and orientation feature are configured to restrain the fiducial in six degrees of freedom, including both +and −x-directions, both +and −y-directions and both +and −z-directions.

43. The device wherein the position and orientation feature is concave and the compliance feature is convex.

44. The device wherein at least one of the compliance feature and the position and orientation feature comprises a latch.

45. The device wherein the latch provides a biasing force with a resultant motion of the fiducial being toward at least one surface of the aimer arm and away from the distal end of the aimer arm.

46. The device wherein the latch biases the fiducial toward a portion of the aimer arm that is away from the latch.

47. The device wherein the latch comprises a latch ramp that engages a fiducial ramp of the fiducial that is complementary in shape to the latch ramp.

48. The device wherein the latch ramp and the fiducial ramp comprise edges or non-planar surfaces.

49. The device wherein the latch extends from the aimer arm on an exterior of the fiducial and contacts an outer surface of the fiducial.

50. The device wherein the latch extends from the aimer arm into an interior of the fiducial and contacts an inner surface of the fiducial.

51. The device wherein the latch is mounted to and extends from the aimer arm.

52. The device wherein the latch is mounted to an exterior of the aimer arm.

53. The device wherein the latch is located in a slot in the aimer arm and is mounted thereto with a fastener.

54. The device wherein the latch is integrally formed with the fiducial.

55. The device wherein the latch is offset from a plane of symmetry for at least one of the fiducial and aimer arm to provide asymmetry and a twisting moment.

56. The device wherein the latch extends from the aimer arm and is configured to provide the twisting moment on the fiducial.

57. The device wherein the latch is cantilevered and recessed within said at least one of the fiducial and aimer arm.

58. The device wherein the latch consists a single cantilever at a proximal end thereof.

59. The device wherein the latch comprises a first cantilever at a proximal end thereof and a second cantilever at a distal end thereof.

60. The device wherein a first cantilever length differs from a second cantilever length such that the first and second cantilever lengths are asymmetrical.

61. The device wherein the latch comprises a pair of cantilevered latches.

62. The device wherein the pair of cantilevered latches are side by side.

63. The device wherein the latch comprises a torsional spring.

64. The device wherein the torsional spring comprises a rotational axis, a recessed position within the aimer arm, and extended position extending away from the aimer arm, and the torsional spring is configured to rotational deflect the fiducial toward the aimer arm.

65. The device wherein the torsional spring is configured to engage a fiducial ramp on an interior of the fiducial.

66. The device wherein the engaged and released positions do not require a separate fastener to attach and detach, respectively, the fiducial with respect to the aimer arm.

67. A device for surgical navigation, the device comprising:
a fiducial comprising a body has a length L, a width W, a height H, a hollow cavity with a cavity length, cavity width and cavity height, the body has an optically detectable portion configured to be optically tracked, and the body has an open front, an open back and side walls; and
an attachment mechanism configured to slidably and releasably couple the body to an aimer arm.

68. The device wherein the attachment mechanism comprises a retaining wire.

69. The device wherein the retaining wire moves with the fiducial when the fiducial moves between engaged and released positions.

70. The device wherein the attachment mechanism comprises a strap.

71. The device wherein the strap is elastic.

72. The device wherein the strap has an end that is configured to be coupled to the fiducial.

73. The device wherein the strap is coupled to a finger tab.

74. The device wherein the strap is molded and integrally formed with the fiducial from a same material.

75. The device wherein the strap and the fiducial are coupled together from different materials.

76. The device wherein the strap has two strap sides.

77. The device wherein each of the two strap sides is configured to extend along only one side of the aimer arm.

78. The device wherein each of the two strap sides is configured to cross and extend along two or more sides of the aimer arm.

79. The device wherein the strap comprises an additional fiducial adjacent a proximal end thereof.

80. The device wherein the strap comprises anchors that are coupled to the fiducial.

81. The device wherein the anchors are flush with interiors of sidewalls of the fiducial.

82. The device wherein the strap is recessed in slots in the sidewalls.

83. A device for a navigation system for robotic surgery with an aimer arm for external optical tracking, the device comprising:
a fiducial comprising an optically detectable portion configured to be optically tracked; and
an attachment mechanism configured to slidably and releasably couple the fiducial to the aimer arm in a sliding clearance fit, the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

84. A device for a navigation system for surgery, the device comprising:
an aimer arm for external optical tracking and configured to be mounted to a base surgical component for surgical navigation, and the aimer arm has a proximal end and a distal end;
a fiducial comprising an optically detectable portion configured to be optically tracked; and
an attachment mechanism configured to slidably and releasably couple the fiducial to the aimer arm in a sliding clearance fit, the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

85. The device wherein the attachment mechanism comprises a retaining wire.

86. The device wherein the retaining wire is in a recess of the aimer arm.

87. The device wherein the retaining wire extends for a limited length of the aimer arm and has a bend at a distal end thereof.

88. The device wherein the retaining wire has an unlocked position that corresponds to the released position of the fiducial, and a locked position that corresponds to the engaged position of the fiducial.

89. The device wherein the retaining wire moves with the fiducial when the fiducial moves between the engaged and released positions.

90. The device wherein, in the engaged position, the retaining wire mechanically interferes with the fiducial to secure it against a hard stop on the aimer arm.

91. The device wherein the retaining wire imparts a biasing force against the fiducial to ensure contact with the hard stop.

92. The device wherein the retaining wire acts as a wedge between the fiducial and the aimer arm.

93. The device wherein the attachment mechanism comprises a strap that couples the fiducial to the aimer arm in the engaged position and the strap is removable from the aimer arm.

94. The device wherein the strap is elastic.

95. The device wherein the strap has a strap proximal end that is configured to be coupled to the proximal end of the aimer arm, and a strap distal end that is configured to be coupled to the fiducial.

96. The device wherein the strap is coupled to a finger tab that can be secured to the proximal end of the aimer arm.

97. The device wherein the strap is molded and integrally formed with the fiducial from a same material.

98. The device wherein the strap and the fiducial are coupled together from different materials.

99. The device wherein the strap has two strap sides.

100. The device wherein, in the engaged position, each of the two strap sides extend along only one side of the aimer arm.

101. The device wherein, in the engaged position, each of the two strap sides cross and extend along two or more sides of the aimer arm.

102. The device wherein the strap comprises an additional fiducial adjacent a proximal end thereof, such that when the fiducial is inside a body of a surgical patient, the additional fiducial is visible from outside the body of the surgical patient.

103. The device wherein the strap comprises anchors that coupled to the fiducial.

104. The device wherein the anchors are flush with interiors of sidewalls of the fiducial.

105. The device wherein the strap is recessed in slots in the sidewalls.

106. A device for a navigation system for robotic surgery, the device comprising:
a fiducial configured to be releasably coupled to an aimer arm with an attachment mechanism in a sliding clearance fit, the fiducial comprises an optically detectable portion configured to be optically tracked, and the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

107. The device wherein the attachment mechanism comprises an open side of the fiducial and a cross bar that spans the open side.

108. The device wherein the fiducial is open on its front and rear and, other than the open side with the cross bar, is closed on every other side.

109. The device wherein the fiducial comprises an internal bore height at the cross bar that is greater than a height of the aimer arm at a bend in the aimer arm, such that edges of the cross bar are spaced apart.

110. The device wherein the fiducial comprises a partial shelf for engaging a surface of the aimer arm.

111. The device wherein the partial shelf extends inward from a sidewall of the fiducial.

112. The device wherein the partial shelf consists of a single partial shelf such that the fiducial is asymmetrical.

113. The device wherein the partial shelf is not in contact with or immediately adjacent to the cross bar.

114. The device wherein the attachment mechanism comprises an open side of the fiducial and a partial shelf that extends inward from a sidewall of the fiducial into the open side, and the fiducial does not comprise a cross bar.

115. The device wherein a length of the partial shelf is less than an overall length of the fiducial.

116. The device wherein the fiducial is open on its front and rear and, other than the open side with the partial shelf, is closed on every other side.

117. The device wherein the partial shelf comprise two partial shelves located opposite each other and extending toward each other.

118. The device wherein the aimer arm comprises a shelf clearance adjacent a distal end thereof for accommodating the partial shelf of the fiducial during installation, and the shelf clearance is on only one side of the aimer arm such that the aimer arm is asymmetric.

119. The device wherein the fiducial is restrained in five degrees of freedom, including both +and −x-directions, both +and −y-directions and one z-direction.

120. The device wherein the attachment mechanism comprises at least one inwardly-biased wall for the fiducial.

121. The device wherein the attachment mechanism comprises inwardly-biased walls, and exposed edges of the inwardly-biased walls are spaced apart from each other by an open side in the fiducial.

122. The device wherein a sidewall of the fiducial comprises a deformable feature on an inner surface thereof.

123. The device wherein sidewalls of the fiducial comprise deformable features on inner surfaces thereof.

124. A device for a navigation system for robotic surgery, the device comprising:
an aimer arm for external optical tracking and configured to be mounted to a base surgical component to for surgical navigation, and the aimer arm has a proximal end and a distal end; and
a fiducial releasably coupled to the aimer arm with an attachment mechanism, the fiducial comprises an optically detectable portion configured to be optically tracked, and the fiducial comprises an engaged position wherein the attachment mechanism engages both the aimer arm and the fiducial such that the fiducial is retained on the aimer arm, and a released position wherein the attachment mechanism disengages such that the fiducial is released from the aimer arm.

125. A fiducial for surgical navigation, the fiducial comprising:
a body has a length L, a width W, a height H, a hollow cavity with a cavity length, cavity width and cavity height, the body has an optically detectable portion configured to be optically tracked, and the body has an open front, open back, an open bottom and sidewalls that can flexibly deform.

126. The fiducial wherein exposed edges of the sidewalls are spaced apart from each other by the open front and the open bottom.

127. The fiducial wherein at least one of the sidewalls comprises a deformable feature on an inner surface thereof.

128. The fiducial wherein each of the sidewalls comprises deformable features on inner surfaces thereof.

129. The fiducial wherein the fiducial has sidewalls and at least one of the sidewalls comprises a deformable feature on an inner surface thereof.

130. A fiducial for surgical navigation, the fiducial comprising:
a body has a length L, a width W, a height H and a hollow cavity with a cavity length, cavity width and cavity height, the body has an optically detectable portion configured to be optically tracked, and the body has a tab that is flexible relative to the body, the tab is configured to engage an aimer arm.

131. A fiducial for a navigation system for robotic surgery, the fiducial comprising:
a hollow body configured to be coupled to an end of an aimer arm, and the hollow body comprises:
a pointer integral with and extending from a distal end of the hollow body;
an engagement feature that is integral with a proximal end of the hollow body, and the engagement feature comprises an engaged position configured to engage the aimer arm such that the fiducial is retained on the aimer arm, and a released position wherein the engagement feature disengages the aimer arm such that the hollow body is released from the aimer arm; and
an optically detectable portion on an exterior of the hollow body, and the optically detectable portion is configured to be optically tracked during computer-assisted surgery.

132. The fiducial wherein the hollow body and the engagement feature are configured to directly attach to and directly detach from the aimer arm without a separate retention component.

133. The fiducial wherein the engagement feature comprises a cantilevered, elastic tab with a recess that is configured to engage, in the engaged position, a protrusion on the aimer arm.

134. The fiducial wherein, in the engaged position, the fiducial is substantially linearly and rotationally immobilized relative to the aimer arm until the cantilevered, elastic tab is released from the protrusion to allow the released position.

135. The fiducial wherein the engagement feature further comprises a pair of engagement features that are configured to engage opposite sides of the aimer arm.

136. The fiducial wherein the hollow body is configured such that the aimer arm inserts into the hollow body.

137. The fiducial wherein the pointer is an aimable feature during computer-assisted surgery.

138. The fiducial wherein the hollow body comprises an aperture configured to receive the end of the aimer arm, and the aperture is located between the pointer and the optically detectable portion.

139. The fiducial wherein a top of the hollow body also comprises the optically detectable portion.

140. The fiducial wherein the hollow body slidingly snaps into place in the engaged position.

141. The fiducial wherein the hollow body comprises shoulders configured to engage respective features on the aimer arm.

142. A fiducial for a navigation system for robotic surgery, the fiducial comprising: a hollow body configured to be coupled to a blunt distal end of an aimer arm, and the hollow body comprises:
a pointer integral with and extending from a distal end of the hollow body, such that the pointer is an aimable feature during computer assisted surgery;
an engagement feature that is integral with a proximal end of the hollow body, and the engagement feature comprises an engaged position configured to engage the aimer arm such that the fiducial is retained on the aimer arm, and a released position wherein the engagement feature disengages the aimer arm such that the hollow body is released from the aimer arm; and
an optically detectable portion on an exterior of the hollow body, and the optically detectable portion is configured to be optically tracked during computer-assisted surgery.

143. The fiducial wherein the hollow body and the engagement feature are configured to directly attach to and directly detach from the aimer arm without a separate retention component.

144. The fiducial wherein the engagement feature comprises a cantilevered, elastic tab with a recess that is configured to engage, in the engaged position, a protrusion on the aimer arm; and
in the engaged position, the fiducial is substantially linearly and rotationally immobilized relative to the aimer arm until the cantilevered, elastic tab is released from the protrusion to allow the released position.

145. The fiducial wherein the engagement feature further comprises a pair of engagement features that are configured to engage opposite sides of the aimer arm; and
the hollow body is configured such that the aimer arm inserts into the hollow body.

146. The fiducial wherein the hollow body comprises an aperture configured to receive the end of the aimer arm, and the aperture is located between the pointer and the optically detectable portion; and
a top of the hollow body also comprises the optically detectable portion.

147. The fiducial wherein the hollow body slidingly snaps into place in the engaged position; and
the hollow body comprises shoulders configured to engage respective features on the aimer arm.

148. A device for a navigation system for robotic surgery, the device comprising:
an aimer arm extending from a handle, wherein a distal end of the aimer arm is blunt and has no aimer tip or other aimable feature for computer-assisted surgery;
a fiducial coupled to the distal end of an aimer arm, and the fiducial comprises:
a pointer integral with the fiducial and extending from a distal end of the fiducial, such that the pointer is an aimable feature during computer assisted surgery;
an engagement feature that is integral with the fiducial at a proximal end of the fiducial, the engagement feature comprises an engaged position to engage the aimer arm such that the fiducial is retained on the aimer arm, and a released position wherein the engagement feature disengages the aimer arm such that the fiducial is released from the aimer arm; and
an optically detectable portion on an exterior of the fiducial, and the optically detectable portion is configured to be optically tracked during computer-assisted surgery.

149. The device wherein the fiducial rotates in a single plane to move from the released position to the engaged position.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” “top”, “bottom,” and the like, may be used herein for ease of description to describe one element's or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated degrees or at other orientations) and the spatially relative descriptions used herein interpreted accordingly.

This written description uses examples to disclose the embodiments, including the best mode, and also to enable those of ordinary skill in the art to make and use the invention. The patentable scope is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

It can be advantageous to set forth definitions of certain words and phrases used throughout this patent document. The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, C, A and B, A and C, B and C, and A and B and C.

Also, the use of “a” or “an” is employed to describe elements and components described herein. This is done merely for convenience and to give a general sense of the scope of the invention. This description should be read to include one or at least one and the singular also includes the plural unless it states otherwise.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the claims.

After reading the specification, skilled artisans will appreciate that certain features that are, for clarity, described herein in the context of separate embodiments, can also be provided in combination in a single embodiment. Conversely, various features that are, for brevity, described in the context of a single embodiment, can also be provided separately or in any sub-combination. Further, references to values stated in ranges include each and every value within that range.

1-149. (canceled)

150. A fiducial for surgical device, the fiducial comprising:
a body configured to be coupled to a distal end of the surgical device; and
an optically detectable portion on an exterior of the body, wherein
the optically detectable portion is configured to be optically tracked during computer-assisted surgery,
the body is configured to receive the distal end of the surgical device to directly attach to and directly detach from the surgical device.

151. The fiducial of claim 150, wherein the body is hollow.

152. The fiducial of claim 150, further comprising:
a pointer integral with and extending from a distal end of the body; and
an engagement feature that is integral with a proximal end of the body, wherein the engagement feature comprises an engaged position configured to engage the surgical device such that the fiducial is retained on the surgical device, and a released position in which the engagement feature disengages the surgical device such that the body is released from the surgical device.

153. The fiducial of claim 152, wherein the body and the engagement feature are configured to directly attach to and directly detach from the surgical device without a separate retention component.

154. The fiducial of claim 152, wherein the engagement feature comprises an elastic tab that is cantilevered, and the elastic tab has a recess that is configured to engage, in the engaged position, a protrusion on the surgical device.

155. The fiducial of claim 154 wherein, in the engaged position, the fiducial is substantially linearly and rotationally immobilized relative to the surgical device until the elastic tab is released from the protrusion to allow the released position.

156. The fiducial of claim 152, wherein the engagement feature further comprises a pair of engagement features that are configured to engage opposite sides of the surgical device.

157. The fiducial of claim 152, wherein the body comprises an aperture configured to receive the distal end of the surgical device, and the aperture is located between the pointer and the optically detectable portion.

158. The fiducial of claim 150, wherein at least one of a top and a side of the body comprises the optically detectable portion.

159. The fiducial of claim 150, wherein the body is configured to slidingly snap into place onto the surgical device.

160. The fiducial of claim 150, wherein the body comprises shoulders configured to engage respective features on the surgical device.

161. The fiducial of claim 150, wherein the body has a tab that is flexible relative to the body, and wherein the tab is configured to engage a recess in the surgical device.

162. The fiducial of claim 161, wherein the tab extends into a hollow cavity of the body.

163. The fiducial of claim 161, wherein the fiducial comprises a flange opposite the tab for engaging a surface of the surgical device that is opposite the recess.

164. The fiducial of claim 163, wherein the flange extends inward from a sidewall of the fiducial.

165. The fiducial of claim 163, wherein the flange comprises a single flange such that the fiducial is asymmetrical.

166. The fiducial of claim 150, wherein the body includes an integrated pointer.

167. The fiducial of claim 150, wherein the surgical device includes an aimer arm.

168. A device for a navigation system for computer-assisted surgery, the device comprising:
a handle;
an aimer arm extending from the handle, wherein a distal end of the aimer arm does not include an aimer tip; and
a fiducial coupled to the distal end of the aimer arm, wherein the fiducial includes
a body coupled to the distal end of the aimer arm, wherein the body is configured to receive a distal end of the aimer arm to directly attach to and directly detach from the aimer arm, and
an optically detectable portion on an exterior of the body, wherein the optically detectable portion is configured to be optically tracked during computer-assisted surgery.

169. The device of claim 168, further comprising a pointer integral with the fiducial and extending from a distal end of the fiducial.
